(19) United States
(12) Patent Application Publication
(10) Pub. No.: US 2025/0213856 A1
(43) Pub. Date: Jul. 3, 2025

(54) BIASED TACHY LEAD FOR CONDUCTION SYSTEM PACING

(71) Applicant: Cardiac Pacemakers, Inc., St. Paul, MN (US)

(72) Inventors: Lili Liu, Maple Grove, MN (US); Shantanu Reddy, Minneapolis, MN (US); Alex Javier Sepulveda, Guaynabo, PR (US); James Finley, Minneapolis, MN (US)

(21) Appl. No.: 18/990,078

(22) Filed: Dec. 20, 2024

Related U.S. Application Data
(60) Provisional application No. 63/616,276, filed on Dec. 29, 2023.

(51) Int. Cl. A61N 1/05

(52) U.S. Cl. CPC A61N 1/0565; A61N 1/0563; A61N 1/0573

(57) ABSTRACT

An implantable lead for use with an implantable medical device (IMD) includes a tubular lead body having proximal end and a distal end opposite the proximal end. A proximal connector is located at the proximal end of the lead body and is configured for mechanically and electrically coupling the lead to the IMD. The lead includes a pre-formed distal portion having a first portion extending from the distal end to a first curve. The first portion is located in a first plane. A second portion extends from the first curve to a second curve, wherein the second portion and the second curve are located in a second plane that is generally orthogonal to the first plane. A helical electrode extends distally from the distal end of the tubular lead body and a ring electrode is located along the first portion.

### CROSS REFERENCE TO RELATED APPLICATIONS

This application claims priority to U.S. Provisional Patent Application No. 63/616,276 entitled “BIASED TACHY LEAD FOR CONDUCTION SYSTEM PACING,” filed Dec. 29, 2023, which is hereby incorporated by reference in its entirety.

### TECHNICAL FIELD

The present disclosure relates to medical electrical leads and associated manufacturing methods and methods of use. In particular, the present disclosure relates to implantable medical electrical leads for stimulating the conduction system of the heart.

### BACKGROUND

Cardiac rhythm management systems are useful for electrically stimulating a patient's heart to treat various cardia arrhythmias. Stimulating the native conduction system of the heart, e.g., the bundle of His, the left bundle branch and/or the right bundle branch can simultaneously pace both the right and left ventricles of the heart, potentially avoiding pacing induced dyssynchrony which may occur with right ventricular apex pacing. There is a continuing need for improved conduction system pacing designs.

### SUMMARY

In Example 1, an implantable lead for use with an implantable medical device (IMD), the implantable lead comprising a lead body having proximal end and a distal end opposite the proximal end, a proximal connector at the proximal end of the lead body configured for mechanically and electrically coupling the lead to the IMD, a pre-formed distal portion comprising a first portion extending from the distal end to a first curve, the first portion being located in a first plane, a second portion extending from the first curve to a second curve, wherein the second portion and the second curve are located in a second plane that is generally orthogonal to the first plane, a helical electrode extending distally from the distal end of the lead body, and a ring electrode located along the first portion.

In Example 2, the implantable lead of Example 1, wherein the pre-formed distal portion includes a third portion extending from the second curve to a third curve.

In Example 3, the implantable lead of Example 2, wherein the second portion and the third portion share the second plane.

In Example 4, the implantable lead of Example 2, wherein the third portion is located in a third plane that intersects the second plane.

In Example 5, the implantable lead of any of Examples 1-4, wherein a first conductor extends through a first lead body lumen and is mechanically and electrically coupled to the helical electrode.

In Example 6, the implantable lead of any of Examples 1-5, wherein a second electrical conductor extends through a second lead body lumen and mechanically and electrically coupled to the ring electrode.

In Example 7, the implantable lead of any of Examples 1-6, wherein the second portion includes a shocking coil.

In Example 8, the implantable lead of any of Examples 2-7, wherein the third portion includes a shocking coil.

In Example 9, the implantable lead of any of Examples 7 or 8, wherein a distal end of the shocking coil is located 30-40 mm from the distal end of the lead body.

In Example 10, the implantable lead of any of Examples 1-9, wherein the pre-formed distal portion includes a collar impregnated with a drug or therapeutic compound.

In Example 11, the implantable lead of any of Examples 1-10, wherein the pre-formed distal portion includes an outer diameter that tapers towards the distal end.

In Example 12, the implantable lead of any of Examples 1-11, wherein the first portion is configured to be directed towards a ventricular septum and the second portion or the second curve are configured to be positioned adjacent a posterior right ventricular septal groove.

In Example 13, the implantable lead of any of Examples 1-12, wherein the ring electrode is located 1.0-1.2 cm from the distal end of the lead body.

In Example 14, the implantable lead of any of Examples 1-13, further comprising a stylet lumen configured for removably receiving a stylet.

In Example 15, the implantable lead of any of Examples 1-14, wherein the pre-formed distal portion is configured to have a generally linear arrangement when constrained by a stylet or delivery catheter.

In Example 16, an implantable lead for use with an implantable medical device (IMD), the implantable lead comprising a lead body having proximal end and a distal end opposite the proximal end, a proximal connector at the proximal end of the lead body configured for mechanically and electrically coupling the lead to the IMD, a pre-formed distal portion comprising a first portion extending from the distal end to a first curve, the first portion being located in a first plane, a second portion extending from the first curve to a second curve, wherein the second portion and the second curve are located in a second plane that is generally orthogonal to the first plane, a third portion extending from the second curve to a third curve, a helical electrode extending distally from the distal end of the lead body, and a ring electrode located along the first portion.

In Example 17, the implantable lead of Example 16, wherein the second portion and the third portion share the second plane.

In Example 18, the implantable lead of Example 16, wherein the third portion is located in a third plane that intersects the second plane.

In Example 19, the implantable lead of Example 16, wherein a first conductor extends through a first lead body lumen and is mechanically and electrically coupled to the helical electrode.

In Example 20, the implantable lead of Example 16, wherein a second electrical conductor extends through a second lead body lumen and mechanically and electrically coupled to the ring electrode.

In Example 21, the implantable lead of Example 16, wherein the second portion includes a shocking coil.

In Example 22, the implantable lead of Example 21, wherein a distal end of the shocking coil is located 30-40 mm from the distal end of the lead body.

In Example 23, the implantable lead of Example 16, wherein the first portion is configured to be directed towards a ventricular septum and the second portion or the second curve are configured to be positioned adjacent a posterior right ventricular septal groove.

In Example 24, the implantable lead of Example 16, wherein the ring electrode is located 1.0-1.2 cm from the distal end.

In Example 25, the implantable lead of Example 16, further comprising a stylet lumen configured for removably receiving a stylet.

In Example 26, the implantable lead of Example 16, wherein the pre-formed distal portion is configured to have a generally linear arrangement when constrained by a stylet or delivery catheter.

In Example 27, an implantable medical implantable lead comprising an elongate body defining a proximal portion and a pre-formed distal portion of the implantable lead, the pre-formed distal portion comprising a first portion extending from a distal end to a first curve, the first portion being located in a first plane, a second portion extending from the first curve to a second curve, wherein the second portion and the second curve are located in a second plane that is generally orthogonal to the first plane, a helical electrode extending distally from the distal end, and a ring electrode located along the first portion.

In Example 28, the implantable medical lead of Example 27, wherein the pre-formed distal portion includes a third portion extending from the second curve to a third curve.

In Example 29, the implantable medical lead of Example 27, wherein the first portion is configured to be directed towards a ventricular septum and the second portion or the second curve are configured to be positioned adjacent a posterior right ventricular septal groove.

In Example 30, the implantable medical lead of Example 27, further comprising a shocking coil located 30-40 mm from the distal end.

In Example 31, the pre-formed distal portion of Example 27, wherein the ring electrode is located 1.0-1.2 cm from the distal end.

In Example 32, the pre-formed distal portion of Example 27, wherein the pre-formed distal portion includes an outer diameter that tapers towards the distal end.

In Example 33, a medical method comprising implanting an implantable medical lead within a right ventricle of a patient's heart, including securing a helical electrode extending from a distal end of the implantable medical lead at a mid- or upper septal location on a right ventricular septum such that the helical electrode is capable of stimulating a left bundle branch or a right bundle branch of the patient's heart, and positioning a shocking coil located on a pre-formed portion of the implantable medical lead at a location adjacent a posterior right ventricular septal groove of the patient's heart. The method further comprises coupling the implantable medical lead to an implantable pulse generator.

In Example 34, the medical method of Example 33, wherein the shocking coil is located 30-40 mm from the distal end of the implantable medical lead.

In Example 35, the medical method of Example 34, wherein the pre-formed distal portion of the implantable medical lead includes a first portion extending from the distal end to a first curve, the first portion being located in a first plane, and a second portion extending from the first curve to a second curve, wherein the second portion and the second curve are located in a second plane that is generally orthogonal to the first plane, and wherein the shocking coil is located along the second curve and is spaced 30-40 mm from the distal end of the implantable medical lead.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic diagram of a conduction system pacing (CSP) system, according to some embodiments of this disclosure.

FIG. 2 is a perspective view of a lead, in accordance with embodiments of the subject matter of the disclosure.

FIG. 3 illustrates a perspective view of a lead having a pre-formed distal portion, in accordance with embodiments of the subject matter of the disclosure.

FIG. 4 illustrates a perspective view of a lead having a pre-formed distal portion, in accordance with embodiments of the subject matter of the disclosure.

FIG. 5 is a front view of the lead of FIG. 4, in accordance with an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The present disclosure concerns, among other things, using the heart's specialized conduction system. In particular, the present disclosure concerns medical electrical leads having one or more electrodes configured to be secured in contact with, or proximate, the nerve fibers of the native conduction system, in particular the left and/or right bundle branches of the heart.

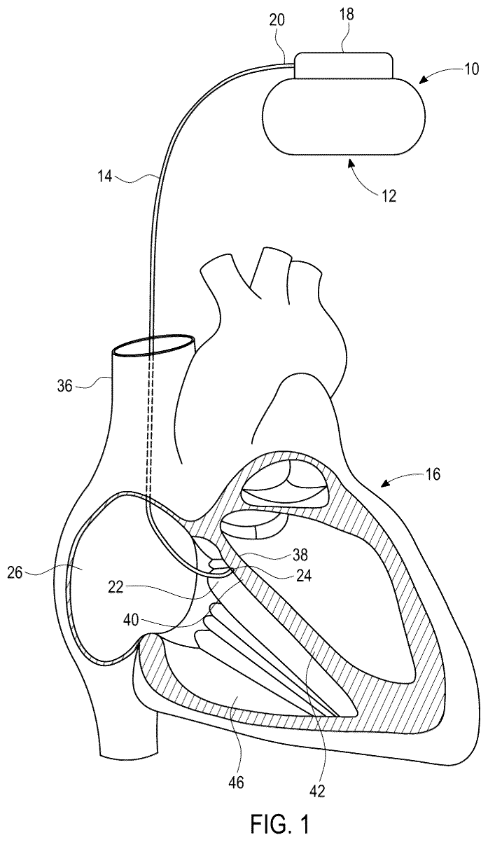

FIG. 1 is a schematic diagram of a conduction system pacing (CSP) system, according to some embodiments of this disclosure. FIG. 1 illustrates a CSP system 10 including an implantable pulse generator 12 and a lead 14. The lead 14 is implanted in a heart 16. The implantable pulse generator 12 can include circuitry for sensing bioelectrical signals and/or delivering electrical stimulation via the lead 14. The implantable pulse generator 12 can include a lead interface 18 (e.g., a header). The lead 14 can include a proximal end 20, a distal end 22, and a fixation element 24 disposed at the distal end 22.

The lead 14 can further include a proximal connector having one or more electrical contacts (not shown) at the proximal end 20, one or more electrical elements (e.g. ring electrodes) at the distal end 22, and one or more electrical conductors (e.g., one or more coils or one or more cable conductors) (not shown) extending within one or more lumens extending within the lead 14 from the electrical contacts to the electrical elements. The lead interface 18 can connect the pulse generator 12 to the electrical contacts at the proximal end 20 of the lead 14 to electrically connect the pulse generator 12 to the electrical elements.

As shown in FIG. 1, the lead 14 is implanted in a right ventricle 46, at a ventricular septum 42, proximate a left bundle branch 38 and/or right bundle branch 40 of the specialized conduction system. The lead 14 operates to convey electrical signals between the target nerve(s), e.g., the left bundle branch 38 and/or the right bundle branch 40 and the implantable pulse generator 12. In some embodiments, the lead 14 can enter the vascular system through a vascular entry site (not shown) formed in a wall of the left subclavian vein (not shown), extend through the left brachiocephalic vein (not shown) and the superior vena cava 36, to the right ventricle 46. Other suitable vascular access sites may be used in various other embodiments.

The fixation element 24 can fix the lead 14 to cardiac tissue, such as the area of tissue by which the left bundle branch 38 and/or the right bundle branch 40 can be directly stimulated. In some embodiments, the fixation element 24 can be electrically coupled to the implantable pulse generator 12 by, for example, one of the electrical conductors, such as a coil, extending to the proximal end 20 of the lead 14 for interfacing with the lead interface 18. As such, the fixation element 24 can mechanically and electrically couple the lead 14 to the tissue and facilitate the transmission of electrical energy from the conduction system in a sensing mode and to conduction system in a stimulation mode. In some embodiments, the fixation element 24 is a fixed fixation element, such as helix fixed to the lead 14. Such a fixation element 24 can be deployed by rotating the lead 14 itself to implant the fixation element 24 into the tissue. The use of the active fixation element for the fixation element 24 may allow for precise placement of the lead 14. The use of the active fixation element for the fixation element 24 may also provide for mapping capability because the user need not be concerned with accidental entanglement of the helix in the tissue.

While FIG. 1 only shows a single lead connected to the implantable pulse generator 12 and implanted for cardiac stimulation, various other embodiments can have an alternative lead and/or one or more additional leads for sensing bioelectrical activity and/or stimulating other areas of the heart 16.

In some embodiments, as will be discussed in greater detail herein, the CSP system 10 can be capable of both pacing and defibrillation therapies. In such embodiments, the lead 14 can also include one or more high voltage defibrillation electrodes (not shown in FIG. 1) for delivering defibrillation shocks capable of terminating ventricular fibrillation.

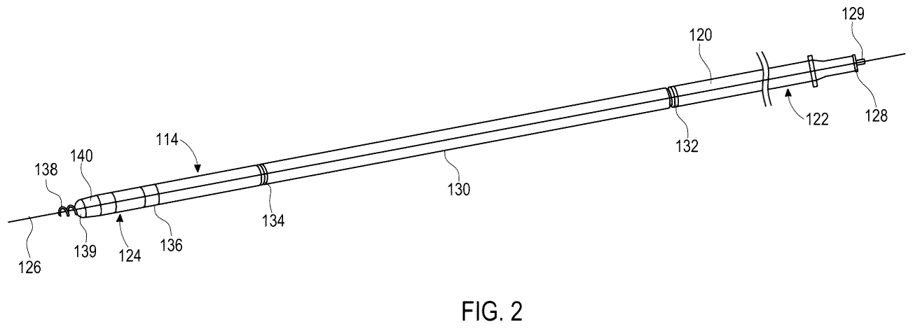

FIG. 2 is a perspective view of a lead 114, in accordance with embodiments of the subject matter of the disclosure. The lead 114 includes a flexible elongate body 120 having a proximal portion 122 and a distal portion 124. The flexible elongate body 120 defines a longitudinal axis 126 of the lead 114. Generally speaking, the proximal portion 122 is dimensioned so as to make up the portion of the lead 114 extending from a pulse generator 12 to the location at which the lead 114 enters the right atrium 26 via the superior vena cava 36, whereas the distal portion 124 is dimensioned to extend within the heart 16 to the location at which the lead 114 is attached to the interior of the heart 16.

The proximal portion 122 includes a connector 128 that is coupled to the proximal end of the flexible elongate body 120. In some embodiments, the connector 128 may include a terminal pin 129 and a ring contact, to electrically connect one or more active electrodes to the implantable pulse generator 12. In some embodiments, the connector 128 is a conventional multipolar connector.

The lead 114 includes a shocking coil 130 positioned along the distal portion 124. The shocking coil 130 is positioned between a first shocking coil coupling 132 and a second shocking coil coupling 134.

A ring electrode 136 is also included along the distal portion 124. The ring electrode 136 is located 1.0 cm-1.2 cm from the distal end 139. The ring electrode is mechanically and electrically connected to the implantable pulse generator 12 by an electrical conductor that is joined to the ring electrode 136. The electrical conductor may travel along a lumen within the flexible elongate body 120 from the ring electrode 136 to the connector 128. The lumen can include more than one electrical conductor. In some embodiments, the lumen is formed in a sidewall of the flexible elongate body 120. In some embodiments, the lumen is located within an interior of the flexible elongate body 120.

The distal portion 124 also includes a fixation element in the form of a helical electrode 138 that extends distally from the distal end 139 of the lead 114. The helical electrode 138 is configured to be rotated in order to fix the lead 114 to a desired portion of the interior of the heart 16. In some embodiments, the helical electrode 138 is electrically active and thus can be used to sense the electrical activity of the heart 16 or to apply a stimulating pulse to the cardiac tissue. This would enable a physician to use the helical electrode 138 to map cardiac tissue and thereby identify an optimal attachment site. In other embodiments, the fixation helix is not electrically active and merely operates as a fixation means. An electrical conductor may travel along a lumen within the flexible elongate body 120 from the helical electrode 138 to the connector 128. In some embodiments, the lumen includes a single electrical conductor. In some embodiments, the lumen can include more than one electrical conductor. The lumen may be positioned within the interior of the flexible elongate body 120 or in a sidewall thereof.

The distal portion 124 also includes a drug collar 140. The drug collar 140 includes an exposed surface and is impregnated with a drug or therapeutic. The drug collar 140 is configured to deliver a drug or therapeutic to a desired tissue within the heart 16. In some embodiments, the drug collar 140 is an overmolded collar. In some embodiments, the drug collar 140 is a pre-molded collar. In some embodiments, the drug collar 140 is omitted.

In order to implant the lead 114 into a desired location in heart or other tissue, the flexible elongate body 120 may be rotated to affix the fixation element to the tissue. In one embodiment, the flexible elongate body 120 may be rotated clockwise to drive the fixation element into the tissue. In another embodiment, the flexible elongate body 120 may be rotated counterclockwise to drive the fixation element into the tissue. In some embodiments, the lead 114 may include a stylet lumen configured to receive a stylet. The stylet provides the lead 114 with rigidity during implantation. As such, during implantation a stylet may be inserted into the lumen to aid implantation of the lead 114 into tissue or advancement of the lead 114 through a patient's vascular system. The lead 114 in FIG. 2 is shown in a generally linear configuration, such as when a stylet is inserted into the stylet lumen.

In some embodiments, the lead 114 tapers from the proximal portion 122 towards the distal end 139. For example, the lead 114 can have a diameter of 8 Fr (2.67 mm) at the shocking coil 130 and have a diameter of 4 Fr to 6 Fr (1.33 mm to 2 mm) at the distal end 139. This tapered configuration may operate to enhance of the distal end 139 into the ventricular septum 42.

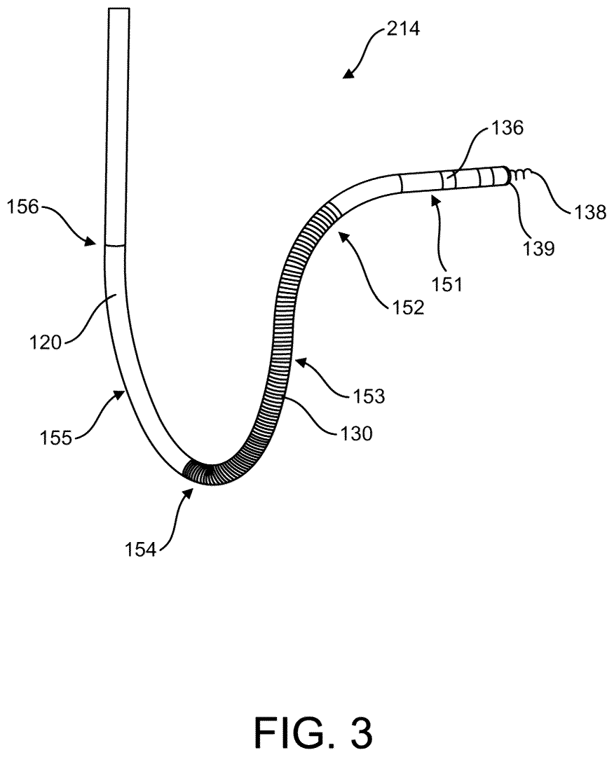

FIG. 3 illustrates a perspective view of a lead 214 having a pre-formed shape, in accordance with embodiments of the subject matter of the disclosure. Lead 214 includes the features of lead 114 discussed above except as otherwise noted. The lead 214 obtains the pre-formed shape when the lead 214 is in an unconstrained configuration. The lead 214 can be constrained externally by an introducer sheath or delivery catheter, or internally by a stylet inserted within a lumen of the lead 214.

In the embodiment illustrated in FIG. 3, the pre-formed shape is located in the distal portion 124 of the lead 214 and directs the helical electrode 138 towards a desired attachment location within the heart 14 while allowing the shocking coil 130 to rest against a portion of the right ventricle 46 when the lead 214 is in an unconstrained configuration. In one embodiment, the helical electrode 138 is configured to be implanted in the ventricular septum 42 while the shocking coil 130 is positioned adjacent a posterior septal groove of the right ventricle 46. The pre-formed shape includes a first portion 151 extending from the distal end 139 to a first curve 152. The first portion 151 is located in a first plane. The first plane is generally orthogonal to a tissue surface during implantation, for example a ventricular septum 42. The first curve 152 can include a curve in the range of in the range of 70-110 degrees.

The pre-formed shape includes a second portion 153 extending from the first curve 152 to a second curve 154. The second portion 153 and the second curve 154 are located in a second plane that is generally orthogonal to the first plane. Generally orthogonal includes planes intersecting or lying at right angles, as well as planes intersecting at angles up to 30 degrees. The second curve 154 allows for the lead 214 to turn back on itself and is substantially U-shaped. Substantially U-shaped includes angles in the range of 150 degrees-210 degrees. The second curve 154 curves in a direction that is opposite of the first curve 152.

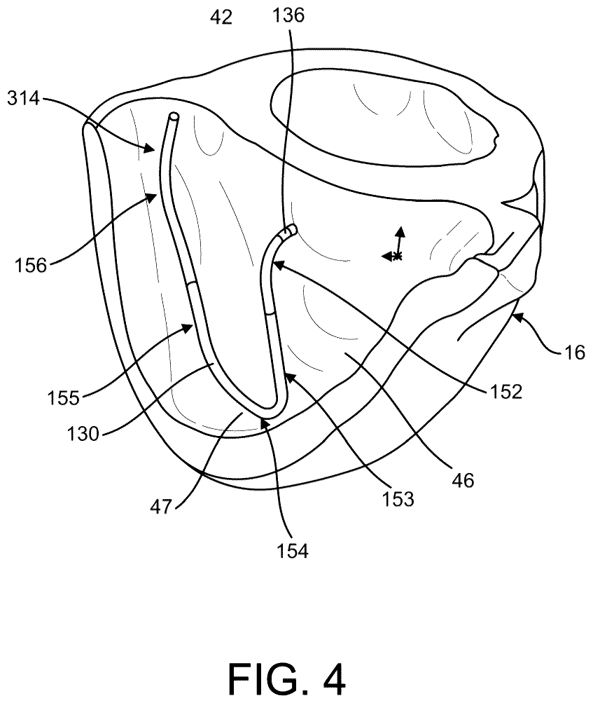

The pre-formed shape also includes a third portion 155 extending from the second curve 154 to a third curve 156. In some embodiments, the second portion 153 and the third portion 155 are both located in the second plane. In some embodiments, as illustrated in FIGS. 4-6, the third portion 155 is located in a third plane that is offset from and intersects the second plane.

As illustrated in FIG. 3, the shocking coil 130 is located along the second portion 153. The shocking coil 130 spans from the first curve 152 to the second curve 154, but does not extend along the third portion 155. In some embodiments, the distal extremity of the shocking coil is located 30-40 mm from the distal end 139 of the lead 114. This spacing is significantly greater than current defibrillation leads used in conduction system pacing applications, which are in the order of 12 mm from the lead tip to the distal-most shocking coil. The inventors of the present disclosure have discovered that the tip-to-coil spacing of 30-40 mm, in conjunction with the preformed curves of the distal portion 124, enable fixation of the distal end 139 in a mid- or high septal wall location while positioning the shocking coil 130 in a location that enhances defibrillation performance. In some embodiments discussed below, the shocking coil 130 is located on the second portion 153 as well as the third portion 155.

FIG. 4 illustrates a perspective view of a lead 314 having a pre-formed distal portion implanted in a heart 16, in accordance with embodiments of the subject matter of the disclosure. Lead 314 is illustrated in an unconstrained shape. During introduction into the heart 16, the lead 314 can be constrained by an introduction sheath or catheter, or by a stylet inserted into a stylet lumen. Lead 314 includes the features of lead 114 discussed above except as otherwise noted.

FIG. 4 illustrates the lead 314 positioned within the right ventricle 46 affixed to the ventricular septum 42 by a helical electrode fixation element (not shown). The distal end of the lead 314 penetrates into the ventricular septum 42 deep enough to allow for simulation of a desired nerve bundle using the helical electrode or ring electrode 136.

The pre-formed distal portion includes a first portion 151 extending from the distal end 139 to a first curve 152. The first portion 151 is located in a first plane. The first plane is generally orthogonal to a tissue surface during implantation, for example the ventricular septum 42. The first curve 152 can include a curve in the range of in the range of 70-110 degrees.

The pre-formed shape includes a second portion 153 extending from the first curve 152 to a second curve 154. The second portion 153 and the second curve 154 are located in a second plane that is generally orthogonal to the first plane. Generally orthogonal includes planes intersecting or lying at right angles, as well as planes intersecting at angles up to 30 degrees. The second curve 154 allows for the lead 214 to turn back on itself and is substantially U-shaped. Substantially U-shaped includes angles in the range of 150 degrees-210 degrees. The second curve 154 curves in a direction that is opposite of the first curve 152.

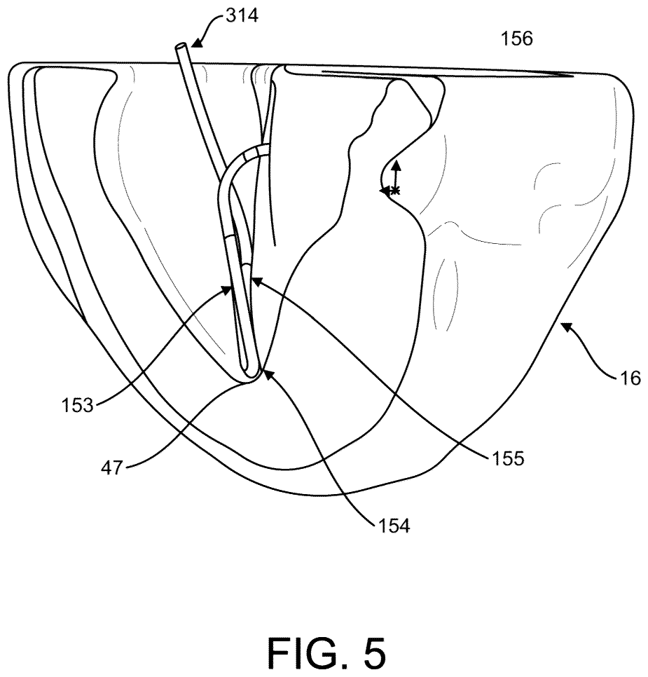

The pre-formed shape also includes a third portion 155 extending from the second curve 154 to a third curve 156. The third portion 155 is located in a third plane that is offset from and intersects the second plane. This can be seen more clearly in FIGS. 5 and 6.

The shocking coil 130 of lead 314 is located on the second portion 153, the second curve 154, and the third portion 155. The shocking coil is located 30-40 mm from the distal end of lead 314 and is located adjacent the right ventricle posterior septal groove 47 of the heart 16.

FIG. 5 is a front view of the lead 314 of FIG. 4, in accordance with an embodiment of the present disclosure. FIG. 5 shows how the third portion 155 extending from the second curve 154 to the third curve 156 lies in a third plane that is offset from and intersects the second plane in which the second portion 153 lies. Additionally shown in FIG. 5, is that a portion of the lead 314 extending proximally from the third curve 156 and into the right atrium extends in a plane that is offset from and intersects the third plane.

The various components of the leads 114, 214, 314 can be made from any known or later developed lead construction materials. For example, the elongate lead body 120 can be made from any flexible, electrically insulative material suitable for human implantation. Exemplary materials for use as the body 120 can include polyurethane, silicone rubber, and co-polymers of both, and can include surface or other treatments (e.g., plasma treatments, lubricious coatings, and the like) based on the functional requirements of the leads 114, 214, 314. The conductors can also be made of any known or later developed lead conductor materials.

Similarly, other components of the leads 114, 214, 314 can be any known or later developed materials. In various embodiments, portions can be made of a non-electrically conductive material such as polyether sulfone (PES), polyurethane-based thermoplastics, ceramics, polypropylene and polyetheretherketone (sold under the brand name PEEK™). Additionally, the connector 128, ring electrode 136, and the helical electrode 138 can be made of any known or later developed conductive material, typically a metal such as Elgiloy, MP35N, tungsten, tantalum, iridium, platinum, titanium, palladium, stainless steel as well as alloys of any of these materials. In various embodiments, ring electrode 136 or helical electrode 138 can include a surface treatment or coating, such as a coating of iridium oxide, to enhance the electrical performance of the ring electrode 136 or the helical electrode 138. Alternatively, the connector 128 and/or the helical electrode 138 can be made of non-electrically conductive materials such as any of the materials described previously.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An implantable lead for use with an implantable medical device (IMD), the implantable lead comprising:
a lead body having proximal end and a distal end opposite the proximal end;
a proximal connector at the proximal end of the lead body configured for mechanically and electrically coupling the lead to the IMD;
a pre-formed distal portion comprising:
a first portion extending from the distal end to a first curve, the first portion being located in a first plane;
a second portion extending from the first curve to a second curve, wherein the second portion and the second curve are located in a second plane that is generally orthogonal to the first plane;
a third portion extending from the second curve to a third curve;
a helical electrode extending distally from the distal end of the lead body; and
a ring electrode located along the first portion.

2. The implantable lead of claim 1, wherein the second portion and the third portion share the second plane.

3. The implantable lead of claim 1, wherein the third portion is located in a third plane that intersects the second plane.

4. The implantable lead of claim 1, wherein a first conductor extends through a first lead body lumen and is mechanically and electrically coupled to the helical electrode.

5. The implantable lead of claim 1, wherein a second electrical conductor extends through a second lead body lumen and mechanically and electrically coupled to the ring electrode.

6. The implantable lead of claim 1, wherein the second portion includes a shocking coil.

7. The implantable lead of claim 6, wherein a distal end of the shocking coil is located 30-40 mm from the distal end of the lead body.

8. The implantable lead of claim 1, wherein the first portion is configured to be directed towards a ventricular septum and the second portion or the second curve are configured to be positioned adjacent a posterior right ventricular septal groove.

9. The implantable lead of claim 1, wherein the ring electrode is located 1.0-1.2 cm from the distal end.

10. The implantable lead of claim 1, further comprising a stylet lumen configured for removably receiving a stylet.

11. The implantable lead of claim 1, wherein the pre-formed distal portion is configured to have a generally linear arrangement when constrained by a stylet or delivery catheter.

12. An implantable medical implantable lead comprising:
an elongate body defining a proximal portion and a pre-formed distal portion of the implantable lead, the pre-formed distal portion comprising:
a first portion extending from a distal end to a first curve, the first portion being located in a first plane; and
a second portion extending from the first curve to a second curve, wherein the second portion and the second curve are located in a second plane that is generally orthogonal to the first plane;
a helical electrode extending distally from the distal end; and
a ring electrode located along the first portion.

13. The implantable medical lead of claim 12, wherein the pre-formed distal portion includes a third portion extending from the second curve to a third curve.

14. The implantable medical lead of claim 12, wherein the first portion is configured to be directed towards a ventricular septum and the second portion or the second curve are configured to be positioned adjacent a posterior right ventricular septal groove.

15. The implantable medical lead of claim 12, further comprising a shocking coil located 30-40 mm from the distal end.

16. The pre-formed distal portion of claim 12, wherein the ring electrode is located 1.0-1.2 cm from the distal end.

17. The pre-formed distal portion of claim 12, wherein the pre-formed distal portion includes an outer diameter that tapers towards the distal end.

18. A medical method comprising:
implanting an implantable medical lead within a right ventricle of a patient's heart, including:
securing a helical electrode extending from a distal end of the implantable medical lead at a mid- or upper septal location on a right ventricular septum such that the helical electrode is capable of stimulating a left bundle branch or a right bundle branch of the patient's heart; and
positioning a shocking coil located on a pre-formed portion of the implantable medical lead at a location adjacent a posterior right ventricular septal groove of the patient's heart; and
coupling the implantable medical lead to an implantable pulse generator.

19. The medical method of claim 18, wherein the shocking coil is located 30-40 mm from the distal end of the implantable medical lead.

20. The medical method of claim 19, wherein the pre-formed distal portion of the implantable medical lead includes:
a first portion extending from the distal end to a first curve, the first portion being located in a first plane; and
a second portion extending from the first curve to a second curve, wherein the second portion and the second curve are located in a second plane that is generally orthogonal to the first plane, and wherein the shocking coil is located along the second curve and is spaced 30-40 mm from the distal end of the implantable medical lead.
